(19) United States
(12) Patent Application Publication
(10) Pub. No.: US 2009/0221872 A1
(43) Pub. Date: Sep. 3, 2009

(54) Endoscope Tip Protector

(75) Inventors: Rodger Liddle, Durham, NC (US); Robert M. Beebe, Raleigh, NC (US)

Correspondence Address: WARD AND SMITH, P.A., 1001 COLLEGE COURT, P.O. BOX 867, NEW BERN, NC, 28563-0867, US

(73) Assignee: MD IDIZ, LLC, Durham, NC (US)

(21) Appl. No.: 12/394,383

(22) Filed: Feb. 27, 2009

Related U.S. Application Data
(60) Provisional application No. 61/067,440, filed on Feb. 28, 2008.

(51) Int. Cl. A61B 1/00

(52) U.S. Cl. 600/121

(57) ABSTRACT

The invention provides an endoscope tip protector for protecting the distal tip of an endoscope and an endoscope assembly including the tip protector affixed thereto. The endoscope tip protector can include a main body with an opening through the main body sized to accept an endoscope sheath and a white surface arranged to face a distal end of an endoscope sheath when the sheath is placed in the endoscope tip protector. Methods of making and using the tip protector and assembly are also provided.

### 1 RELATED APPLICATION

This application claims the priority of U.S. Provisional Patent Application Ser. No. 61/067,440, filed Feb. 28, 2008, the disclosure of which is incorporated by reference in its entirety.

### 2 FIELD OF THE INVENTION

The invention relates to an endoscope tip protector for protecting the distal tip of an endoscope, to an endoscope assembly including the tip protector affixed thereto, and to methods of making and using the tip protector and assembly.

### 3 BACKGROUND OF THE INVENTION

Over the past three decades, endoscopes have been used for medical, industrial and other purposes in which high quality optical viewing is facilitated by a steerable, flexible extension device. These instruments have at one end a lens or optical chip and light connected by a flexible shaft to a control device that allows the operator to manipulate the flexible viewing portion of the instrument (typically on the distal end of the shaft).

Endoscopes are powered electrically and images are viewed through an optical eye piece or projected onto a video or computer screen. Endoscopes are commonly used medically for performing diagnostic or therapeutic procedures such as colonoscopy, esophagogastroduodenoscopy, endoscopic retrograde cholangiopancreatography, endoscopic ultrasound, bronchoscopy, cystoscopy, laparoscopy, and arthroscopy and other minimally invasive procedures. As medical procedures become less invasive, endoscopic approaches are becoming increasingly more common. However, with the improvement in technologies, endoscopes have become more expensive.

Endoscopes are technologically sophisticated and comprised of fragile components that are easily damaged or broken. Endoscope damage is often due to damage of the tip due to puncture at the distal more flexible end of the endoscope or hitting the tip of the scope on the floor or another hard surface thereby damaging the video chip. It has been reported that over 60% of all endoscope repairs are due to this type of damage with an average repair cost of $3,000.00. There is a need in the art for a way to protect endoscope tips from expensive damage.

### 4 BRIEF DESCRIPTION OF THE INVENTION

The invention provides an endoscope tip protector (ETP) for protecting the distal tip of an endoscope as well as an endoscope assembly comprised of the tip protector. In one embodiment, an ETP is provided comprising a main body comprising an opening through the main body sized to accept an endoscope sheath and a white surface arranged to face a distal end of an endoscope sheath when the sheath is placed in the ETP. In another embodiment, an ETP is provided comprising a main body comprising an opening through the main body sized to accept an endoscope sheath and a plug arranged in a distal end of the opening. Methods of making and using the tip protector and assembly are also provided.

### 5 BRIEF DESCRIPTION OF THE DRAWINGS

FIG. 1 illustrates embodiments of endoscope tip protectors with either the central opening extending through all of the body of the ETP (Panel A) or with the central opening not extending throughout the ETP body (Panel B).

FIG. 2 illustrates side views of differently shaped endoscope tip protectors (Panels A-C).

FIG. 3 illustrates one embodiment of an endoscope assembly with an endoscope being inserted into the ETP and a plug at the distal end of the protector.

FIG. 4 illustrates embodiments of endoscope tip protectors with a plug (Panel A) or a cap (Panel B).

FIG. 5 illustrates top views of embodiments of differently shaped openings of the ETP (Panels A-D).

FIG. 6 illustrates a top view of an embodiment of an ETP with different sized openings.

FIG. 7 illustrates top (Panel A), bottom (Panel B), and perspective (Panel C) views of an embodiment of an ETP in which the central opening is off-center relative to the body of the ETP.

FIG. 8 depicts top views of spherical (Panel A) and oblong (Panel B) embodiments of endoscope tip protectors without a central opening (such as for washing).

### 6 DETAILED DESCRIPTION OF THE INVENTION

The invention provides an endoscope tip protector, an endoscope assembly including an endoscope having the ETP affixed thereto, and methods of making and using the ETP and ETP assembly. The ETP may be mounted to or affixed to a distal end of an endoscope to protect the tip. In some embodiments, the ETP may also be configured to facilitate use of the ETP for washing the endoscope between uses. In other embodiments, the ETP may be used to facilitate white light balancing of the endoscope.

6.1 ETP Designs

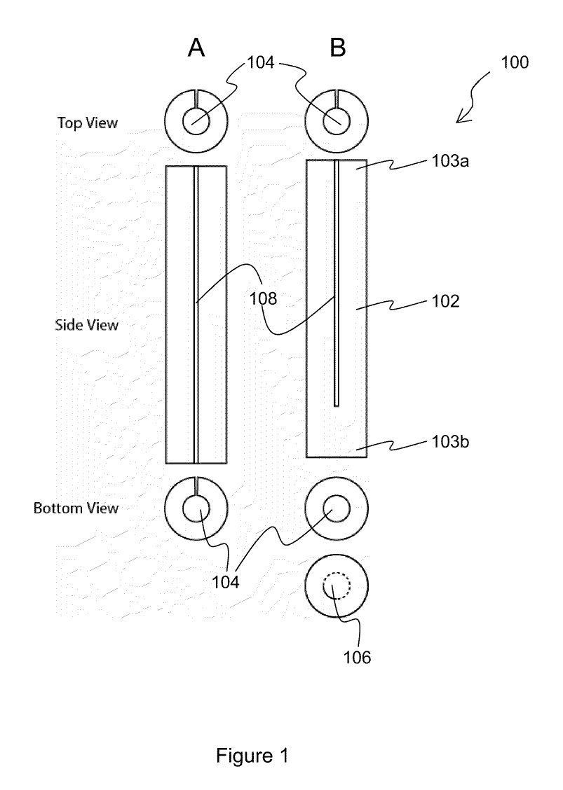

FIGS. 1-8 illustrate several embodiments of the ETP 100. In the embodiment shown in FIG. 1, ETP 100 may include an ETP body 102, an opening 104 extending at least partially through the ETP body 102, a proximal end 103a, a distal end 103b, a slit 108 extending through the ETP body 102 to the opening 104, and a plug 106 for insertion in a distal end of the opening 104.

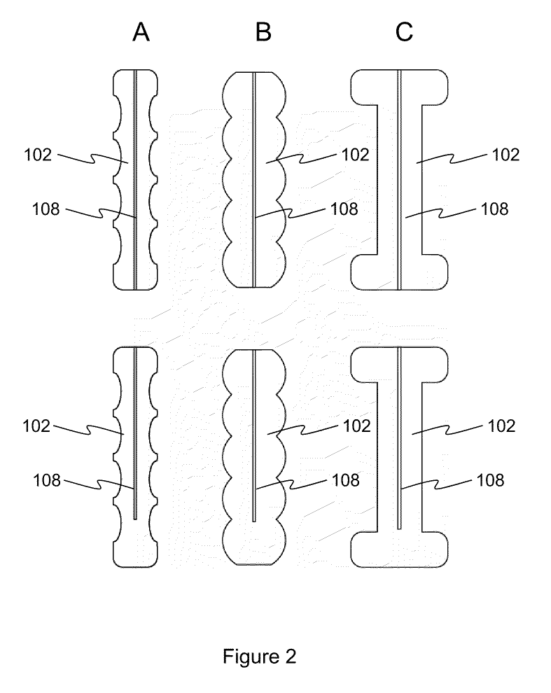

The ETP body 102 may be provided in any of a wide variety of shapes. Examples include cylindrical, spherical, elongated sphere, etc. Many alternatives of a generally cylindrical shape are also possible, such as elongated polygon (such as a multi-sided cylinder), elongated starburst, and the like. Further, the elongated cylinder structure may have indentations and/or undulations along the length of the cylinder, e.g., as shown in FIGS. 2A and 2B. A dumbbell shape, e.g., as shown in FIG. 2C, is also possible. A preferred embodiment of the ETP is a cylinder or a cylinder-like structure.

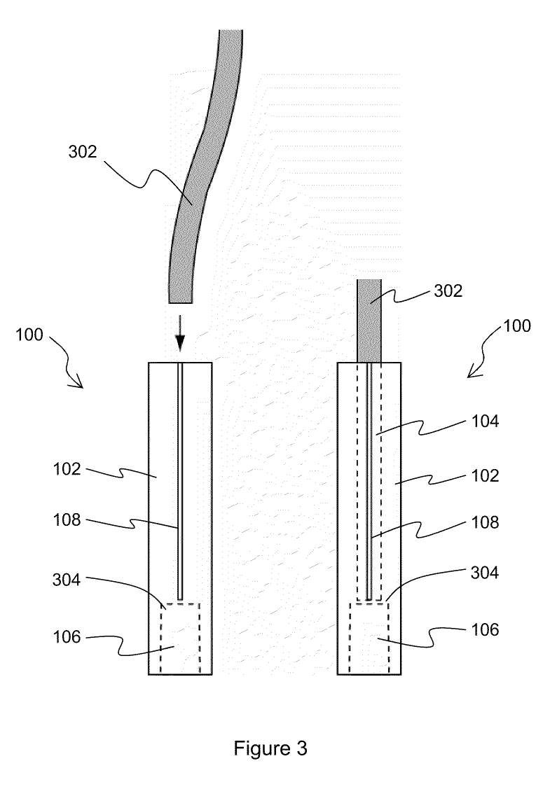

The ETP body 102 includes an opening 104 extending partway or completely through the ETP body 102. Typically, the opening 104 extends lengthwise through an elongated ETP body 102. As illustrated in FIG. 3, the opening 104 is sized for accepting the distal end of an endoscope. The size of opening 104 may vary depending on the type of endoscope used. The size may be selected to fit any type of endoscope. Examples include endoscopes for colonoscopy, upper endoscopy, endoscopic retrograde cholangiopancreatography, endoscopic ultrasound, bronchoscopy and cystoscopy, and otolaryngology procedures. The opening 104 is preferably sized so that it is relatively easy to slide it onto the tip of the endoscope 302, but tight enough to remain firmly in place on the tip of the endoscope. The size may be selected to affect a snug fit on the endoscope. In one cylindrical embodiment, the endoscope has the following dimensions: 10 cm length; 3 cm outside diameter; 1.5 cm luminal diameter (luminal diameter may vary from 0.5 cm to 2 cm). The walls of the opening 104 may comprise a white surface arranged to face the distal end or tip of the endoscope (or endoscope sheath) when the ETP is placed thereon.

Again referring to FIG. 3, the distal end of opening 104 may be closed by an externally inserted plug 106 or a plug that is formed of the same material as the ETP, e.g., using perforations (see plug 106 in FIG. 1).

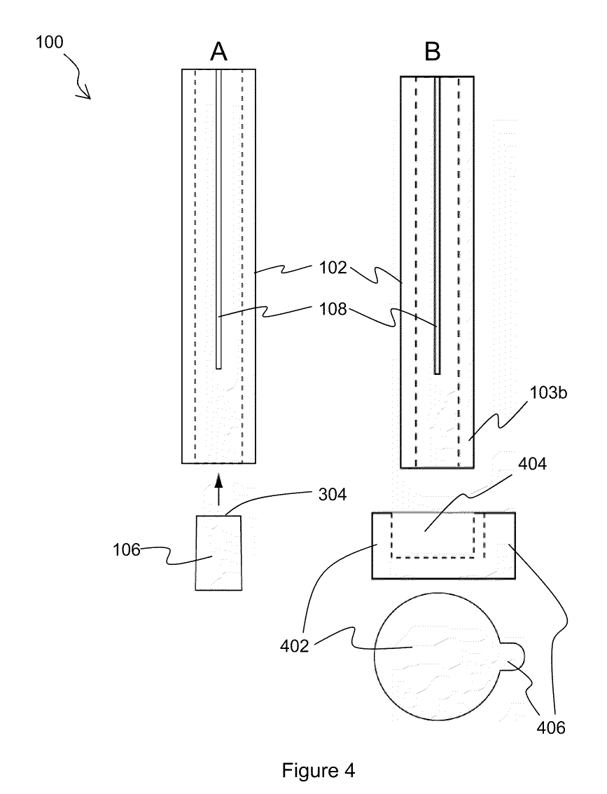

FIG. 4A illustrates a preferred embodiment in which plug 106 is shown and the proximal surface 304 of the plug 108 is opaque white. In this embodiment, the plug 106 is disposed in the distal end of the endoscope such that when inserted, the plug 106 will prevent the endoscope tip from extending all the way through the ETP body 102. As discussed further below, the plug 106 may have a white surface exposed to the endoscope tip when placed on the endoscope to facilitate white light balancing while the ETP 100 is mounted on the endoscope. Various alternatives to a plug are also possible, such as a cap or other means for covering the distal end of the opening 104.

FIG. 4B also depicts a cap 402 which includes a tab 406 for ease in removal of the cap. The cap includes an opening 404 having a width sufficient to fit over a distal end 103b of the ETP body 102. Where a cap is used, the cap may include a tab 406, ridged structure, grippable surface, or other device to facilitate manual removal of the cap. The plug 106 and the cap 402 are not exclusive and therefore, one ETP may have a plug, a cap, and/or other means for covering the distal end.

In embodiments in which the opening 104 does not extend all the way through the ETP body 102, there is no need for a plug. However, the surface exposed to the endoscope may nevertheless be white to facilitate white light balancing. Further, the material may be scored or perforated to permit the endoscope tip to be forced through the end of the ETP body 102 to facilitate use of the ETP body 102 for washing.

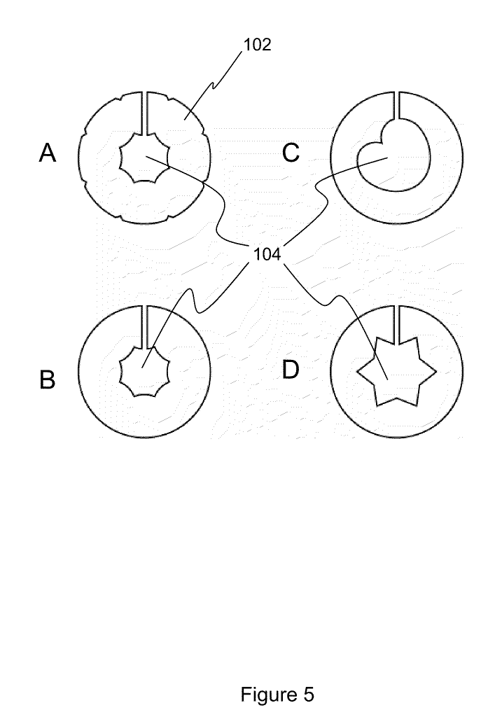

FIG. 5 illustrates a lobed opening 104(A and B), a heart-shaped opening 104(C), and a ridged opening 104(D)—the latter forming a star-shaped opening. It will be appreciated that a wide variety of openings is possible within the scope of the invention.

FIG. 5A also illustrates longitudinal lobes along the exterior of the ETP body 102, the purpose of which may include ease of gripping, manufacture forming, and/or aesthetic distinctive design.

The ETP body 102 may also include a slit 108. When present, the slit 108 is arranged to facilitate placement of the ETP 100 on the distal end of an endoscope sheath. For example, the ETP body 102 may include a generally linear slit 108 extending from the proximal end 103a towards the distal end 103b to allow easy placement of the ETP 100 on an endoscope tip. In some embodiments, the slit 108 may extend through the entire length of the ETP body 102, e.g., as shown in FIG. 1A. In some embodiments, the linear slit 108 may extend partially through the length of the ETP, e.g., as shown in FIG. 1B. FIG. 2 also shows various embodiments of the ETP in which the slit 108 extends through the entire length of the ETP body 102 (embodiments shown in top portion), or only partially through the ETP body 102 (embodiments shown in bottom portion).

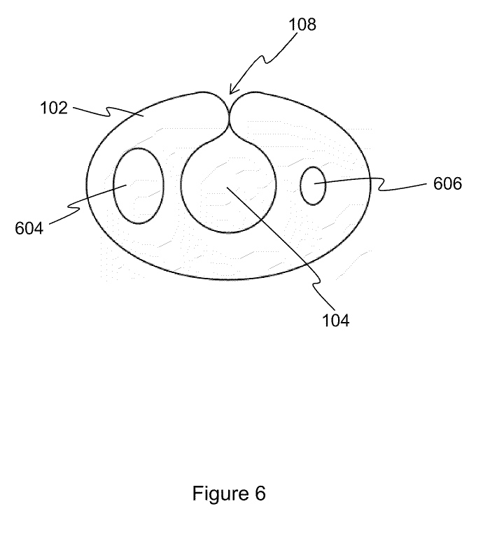

FIG. 6 illustrates another embodiment of the invention, including an ETP body 102, an opening 104 extending through the ETP body 102, and a slit 108 extending through the ETP body 102 to the opening 104. This embodiment also includes additional openings, for example large openings 604 and small openings 606, extending into or through the ETP body 102. The openings 104, 604, 606 may, as in other embodiments described herein, be closed with a cap and/or a plug. In operation, the opening 104 is particularly useful for washing the endoscope. The endoscope may be placed in the opening 104 by inserting it laterally through slit 108. The openings 604, 606 are provided for protection of the endoscope tip. They are preferably differentially sized so that the ETP may be used to protect scopes having tips of different sizes. The number of openings 604, 606 can vary in a particular ETP and can range from 0, 1, and up to many. In a preferred embodiment, two openings 604,606 are found in one ETP.

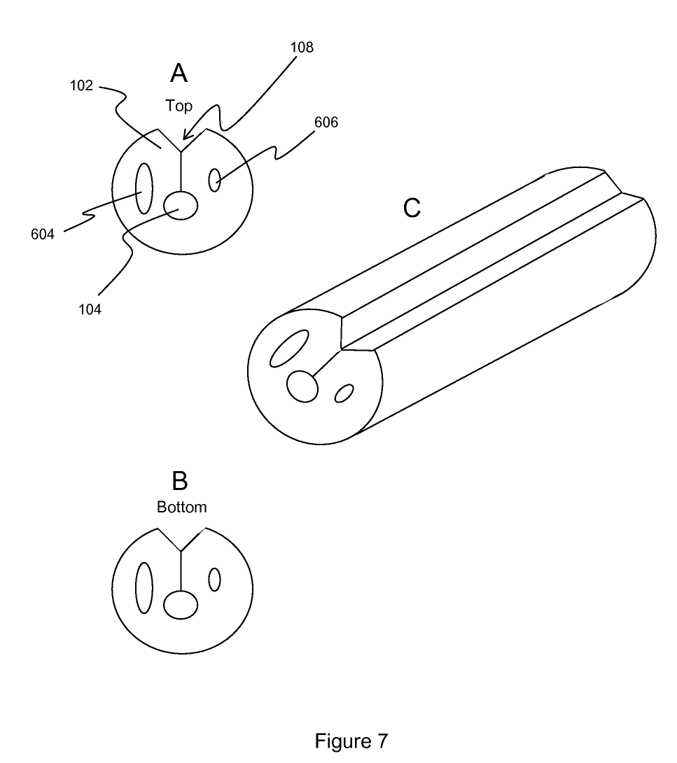

FIG. 7 illustrates yet another embodiment of the invention, including the ETP body 102, a V-shaped slit 108, and openings 104, 604, 606. In this embodiment, the central opening 104 is slightly off-center in relation to body 102. One non-limiting purpose of this design is to enable a thinner wall between the central opening 104 and the outer wall of body 102 on the side opposite the V-shaped slit 108, thereby facilitating placement and/or washing of an endoscope inserted therein. The V-shape of 108 is included in order to, among other things, facilitate placement of an endoscope inserted into the ETP from a lateral direction (wherein the V nests and holds the endoscope sheath prior to placement into the ETP) or otherwise ease opening of the slit 108 by the user (such as through thumb placement and opening).

In general, the ETP can have the dimensions required for the endoscope(s) it is protecting. In an exemplary embodiment, the ETP body 102 may have a length of 125 mm and a width of 50 mm. The diameter of the central opening 104 may be approximately 10 mm, the large opening 604 may have dimensions of 7 mm×18 mm, and the small opening 606 may have dimensions of 3 mm×7 mm. The depth of the V-shaped slit 108 may be 10 mm. It is understood that the various dimensions of the ETP can vary without departing from the scope of the invention.

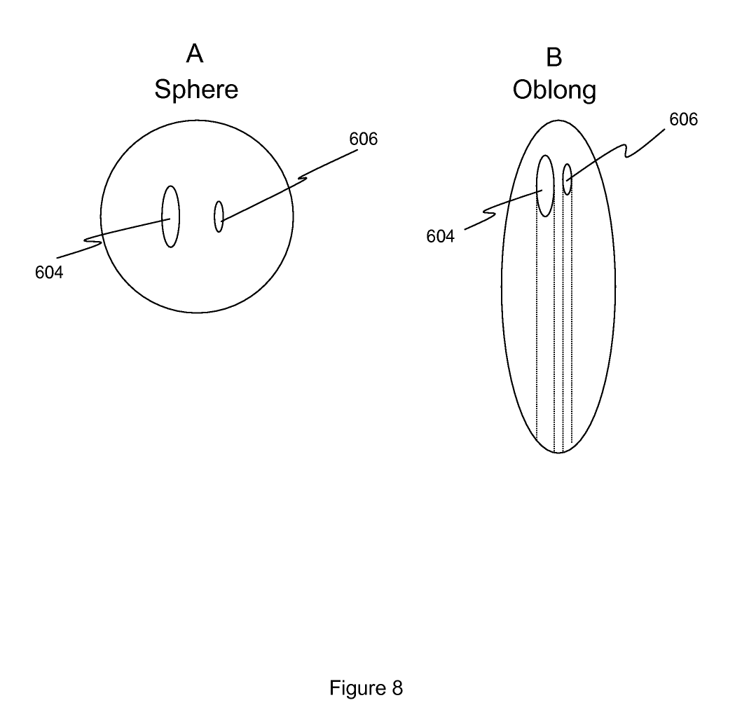

FIG. 8 depicts both a spherical (A) and an oblong (B) ETP. In this embodiment, the ETP is not comprised of a central washing bore or opening. Openings 604, 606 are still present for accepting different sized endoscope tips, but the central washing bore is not needed. In this case, the entire device can be folded over so that the body of the device itself can be used for washing at least one endoscope.

In general, the ETP may have the dimensions required for the endoscope(s) it is protecting. In one exemplary spherical embodiment, the ETP may have a width diameter range from 40-125 mm, the large opening 604 may have dimensions of 7 mm×18 mm, and the small opening 606 may have dimensions of 3 mm×7 mm. In one exemplary oblong embodiment, the ETP may have a length of 125 mm, a width diameter range from 40-100 mm, the large opening 604 may have dimensions of 7 mm×18 mm, and the small opening 606 may have dimensions of 3 mm×7 mm. It is understood that the various dimensions of the ETP can vary without departing from the scope of the invention. For example, in a non-limiting example, the large opening 604 may have dimensions in the range of 6-8 mm×16-20 mm and the small opening 606 may have dimensions in the range of 2-5 mm×6-8 mm.

### 6.2 Materials and Manufacturing

The ETP 100 may be manufactured using standard processes for molding or shaping materials. The size and nature of the material are selected to protect an endoscope from damage. For example, the size and nature of the material may be selected to protect an endoscope from damage when dropped from a 5 foot height which approximates the height of a procedure table.

The ETP body 102, plug 106, and/or cap 402 may be made of soft foam or other comparable material. Material should be selected that protects the endoscope tip from one or more sources of damage, such as damage caused by needle puncture, laceration, compression, concussion or other external damage. Preferred foams are those which do not leave a residue on the endoscope. In some embodiments, the material selected also has absorption characteristics that facilitate its use as a sponge for washing the instrument after a use. In one embodiment, the ETP body 102, plug 106 and/or cap 402 may be made of rubber, such as styrene-butadiene rubber. In a more preferable embodiment, the ETP body 102, plug 106 and/or cap 402 is made of polyurethane foam that is either flexible or rigid. More preferably, the ETP body 102, plug 106 and/or cap 402 is made of flexible, polyurethane foam. Alternative embodiments may include high density foam, rebond foam, supreem foam, memory foam, latex rubber foam, high resilience foam, Evlon foam, dry fast foam, closed cell foam and other polyurethane foams. Other embodiments may also include biodegradable foams, such as polyurethane foams, silicone foams, neoprene and EPDM foams, and rubber melamine foams. Most preferably, the ETP body 102, plug 106 and/or cap 402 is made of medical grade foam. In a preferred embodiment, the entire ETP body 102, plug 106 and/or cap 402 includes a white material or a material having a white surface. The material selected is preferably devoid of latex.

It will be appreciated from the discussion above that a wide variety of combinations of the elements of the invention are possible within the scope of the invention. In one embodiment, the ETP body 102 includes a cylinder with a central opening 104 and slit 108 extending the entire length of the ETP body 102. In another embodiment, the ETP body 102 includes a cylinder with a central opening extending from the proximal end 103a towards the distal end 103b, but not penetrating the distal end 103b, so the distal end is closed. The distal end may provide a white surface for white light balancing. The distal end may include perforations for permitting the endoscope tip to be forced through the distal end to facilitate washing. In yet another embodiment, the ETP body 102 includes a cylinder with a central opening 104 extending from the proximal end 103a towards the distal end 103b, but not penetrating the distal end 103b.

The distal end may be perforated or partially perforated (e.g., see FIG. 1B, 106) so the endoscope tip can be pushed through the distal end. In the partially perforated embodiment, the endoscope tip can be pushed through the distal end leaving a plug 106 or end attached to the ETP. In the fully perforated version, the perforated plug can be pushed out and need not remain attached. In any of the various embodiments described herein, it is preferred that at least the surface of the ETP body 102, plug 106 and/or cap 402 facing the distal end (relative to the operator) of the endoscope is white. In other embodiments, the entire ETP body 102, plug 106 and/or cap 402 is white.

6.3 Packaging

The ETP may be individually wrapped in a clean plastic wrapper. One embodiment of the plastic wrapping includes a plastic, easy to open, tear-off portion making the ETP 100 easily accessible. In some embodiments, individual ETPs may be stored in a dispenser that may also facilitate distribution. The packaging is preferably devoid of latex.

6.4 Use of the Tip Protector

In use, the ETP is placed on the distal end of the endoscope sheath. The tip protector may remain in place when the endoscope is stored (e.g., in a container, on a hanger or in a closet) and/or when the endoscope is transported to the place of use (e.g., procedure room in the case of medical procedures). The ETP is removed from the endoscope prior to insertion of the endoscope into the subject. Following completion of the procedure, the ETP may be replaced on the scope, protecting the scope from sharp instruments such as needles and biopsy forceps, and safely transported back to the cleaning room for reprocessing.

6.4.1 Use of ETP for Washing

The ETP body 102 may also be used for washing the endoscope sheath. Washing may be facilitated by the opening 104 which may be repetitively moved along the sheath to create a washing action. Alternatively, other openings in the ETP body 102 may be used for washing if the ETP is folded over, such as described above with reference to FIG. 8. The ETP may also have an easy-to-grip design, e.g., as illustrated in FIG. 2, to facilitate handling by the user during washing.

6.4.2 Use of ETP for White Light Balancing

As noted above, in some embodiments, the portion of the ETP exposed to the imaging components may be white in order to facilitate white light balancing. At the beginning of each endoscopic procedure, the contrast/color settings of the endoscope and power source are typically adjusted by the operator or assistant by placing the endoscope tip against a white surface, thus resetting the color parameters of the system. This procedure is referred to as “white light balancing” and is recommended by the endoscope manufacturers. The white feature of the ETP is useful for permitting white light balancing without requiring removal of the ETP. In another embodiment, the inner wall of the opening through the ETP is white, and white light balancing is achieved by closing the opening manually by using pressure, e.g., manual pressure, to force together opposite sides an inner wall of the opening.

### 7 CONCLUDING REMARKS

The foregoing detailed description of embodiments refers to the accompanying drawings, which illustrate specific embodiments of the invention. Other embodiments having different structures and operations do not depart from the scope of the present invention.

The term “the invention” is used with reference to certain specific examples of the many alternative aspects or embodiments of the applicants' invention set forth in this specification, and neither its use nor its absence is intended to limit the scope of the applicants' invention or the scope of the claims.

This specification is divided into sections for the convenience of the reader only. Headings should not be construed as limiting of the scope of the invention.

It will be understood that various details of the present invention may be changed without departing from the scope of the present invention. Furthermore, the foregoing description is for the purpose of illustration only, and not for the purpose of limitation, as the present invention is defined by the claims as set forth hereinafter.

We claim:

1. An endoscope tip protector comprising:
(a) a main body comprising an opening at least partially through the main body sized to accept an endoscope sheath; and
(b) a white surface arranged to face a distal end of an endoscope sheath when the sheath is placed in the endoscope tip protector.

2. The endoscope tip protector of claim 1 further comprising a slit through the main body of the endoscope tip protector, wherein the slit extends partially through the length of the body or through the entire length of the body.

3. The endoscope tip protector of claim 1 wherein the opening through the main body is used for washing an endoscope.

4. The endoscope tip protector of claim 1 wherein the opening through the main body is off-center relative to the main body.

5. The endoscope tip protector of claim 1 further comprising one or more additional openings through the main body differently sized to accept differently sized endoscopes.

6. The endoscope tip protector of claim 1 wherein the tip protector can be folded over in order to provide for washing at least one endoscope.

7. The endoscope tip protector of claim 1 further comprising a plug arranged in a distal end of the opening.

8. The endoscope tip protector of claim 7 wherein the white surface is arranged on the plug.

9. The endoscope tip protector of claim 1 further comprising a cap fitted on a distal end of the main body.

10. The endoscope tip protector of claim 1 having a shape selected from the group consisting of: cylindrical, spherical, elongated sphere, elongated polygon, elongated starburst, elongated cylinder with indentations and/or undulations along the length of the cylinder, and dumbbell.

11. The endoscope tip protector of claim 1 wherein the ETP is provided in a size selected to protect an endoscope for a procedure selected from the group consisting of colonoscopy, upper endoscopy, endoscopic retrograde cholangiopancreatography, endoscopic ultrasound, bronchoscopy, cystoscopy, and otolaryngology.

12. The endoscope tip protector of claim 1 wherein the distal end of the opening is closed by an externally inserted plug.

13. The endoscope tip protector of claim 1 wherein the distal end of the opening is closed by a plug that is formed of the same material as the ETP.

14. The endoscope tip protector of claim 1 wherein the distal end of the opening is closed by a plug that is formed by perforations.

15. The endoscope tip protector of claim 1 wherein the body comprises a foam.

16. The endoscope tip protector of claim 15 wherein the foam has absorption characteristics that facilitate its use as a sponge for washing the endoscope after a use.

17. The endoscope tip protector of claim 1 wherein the opening comprises a shape selected from the group consisting of longitudinally ridged, longitudinally lobed, and longitudinally heart shaped.

18. An endoscope assembly comprising an endoscope and the endoscope tip protector of claim 1, wherein the endoscope tip protector is placed on a distal end of the endoscope.

19. A method of white light balancing an endoscope comprising:
(a) placing the endoscope tip protector of claim 1 on a distal end of the endoscope; and
(b) white light balancing the endoscope.

20. An endoscope tip protector comprising:
(a) a main body comprising an opening through the main body sized to accept an endoscope sheath; and
(b) a plug arranged in a distal end of the opening.

21. The endoscope tip protector of claim 20 comprising a white surface arranged on the plug.

22. An endoscope assembly comprising an endoscope and the endoscope tip protector of claim 21, wherein the endoscope tip protector is placed on a distal end of the endoscope.

23. A method of white light balancing an endoscope comprising:
(a) placing the endoscope tip protector of claim 20 on a distal end of the endoscope; and
(b) white light balancing the endoscope.

24. A method of white light balancing an endoscope comprising:
(a) placing the endoscope tip protector of claim 1 on a distal end of the endoscope; and
(b) closing the opening manually by using pressure to force together opposite sides of an inner wall of the opening.
